(19) United States
(12) Patent Application Publication
(10) Pub. No.: US 2019/0124024 A1
(43) Pub. Date: Apr. 25, 2019

(54) CATEGORIZED ELECTRONIC MESSAGING

(71) Applicant: CLINOMICSMD LTD., Leduc (CA)

(72) Inventors: Colin OBERG, Kelowna (CA); Thomas PEEBLES, Grande Prairie (CA); Felix SOIBELMAN, Edmonton (CA)

(21) Appl. No.: 15/789,784

(22) Filed: Oct. 20, 2017

(51) Int. Cl. H04L 12/58

(52) U.S. Cl. CPC H04L 51/12; G16H 80/00; H04L 51/02; H04L 51/14

(57) ABSTRACT

Methods, systems, and program products for electronic messaging involve: storing a permitted message category set in association with a receiver account; communicating with a sender computer device accessing the sender account to generate a message category input graphical user interface (GUI) element configured to receive a message category input limited to a member of the permitted message category set, for association with a message addressed to the receiver account; receiving from the sender computer device, and storing in the database, the message category input in association with the message; and communicating with a receiver computer device accessing the receiver account to generate on a display device of the receiver computer device a plurality of message category GUI elements and a message indicator GUI element indicative of the message in association with the message category GUI element corresponding to the message category input.

### FIELD OF THE INVENTION

The present invention relates to electronic messaging between computer devices via a communication network.

### BACKGROUND

Physicians work within health care teams that include other physicians (e.g., doctors, nurse practitioners, residents and medical students), and other professionals (e.g., nurses, pharmacists, therapists, administrative staff etc.). Efficient, convenient, and secure communication among the team members and patients is essential.

Electronic mail (email) is currently the most popular mode of text-based electronic communication in many industrial settings. Conventional email systems deliver email messages from a sender to a recipient using an email address scheme consisting of the user's name followed by the “@” symbol and a mail server domain. The sender uses a client program to prepare and send the email message, which consists of a message body and a message header. The message header is structured into fields that includes a subject line. After the email message is sent, it is received and held by the recipient's mail server. The recipient uses another client program that generates a graphical user interface for an “inbox” displaying a list of message header information for all received email messages. The recipient may select the message header information of one of the email messages to display the message body of that email message.

Conventional email systems have shortcomings. First, any user can send an email message to the known email address of any other user. Thus, the recipient's inbox may receive unsolicited email messages, which can be a nuisance and pose security threats. As an example in the health care context, a physician may not want to receive unsolicited email messages from unknown persons seeking medical advice.

Second, the subject line of the email message header may not accurately describe the message body. As an example in the health care context, a physician receiving an email message with the subject line “Re: patient X” in the inbox, must actually read the message body to know whether the email message actually concerns patient X, and whether it requires timely attention.

Third, conventional email systems may be configured to automatically forward an email message to a designated email address, and to automatically send a reply message to a sender's email address. However, these features apply universally to all received email messages, which may be undesirable. As an example in the health care context, a physician might receive an email message from a family physician requesting a patient consultation, which should be forwarded to office staff responsible for booking patient appointments. The physician might receive another email message with a radiology report, which should be forwarded to other health care providers (e.g., a family physician and medical residents).

Accordingly, there is a need in the art for improvements to electronic messaging that address the shortcomings of conventional email systems.

### SUMMARY OF THE INVENTION

In one aspect, the present invention comprises a method for electronic messaging between client accounts including a sender account and a receiver account. The method is implemented using a server computer device, which is operatively connected to a computer-readable database, and to a communications interface for communicating via a communication network with computer devices. The method comprises the steps of:
(a) storing in the database in association with the receiver account, a permitted message category set;
(b) communicating with a sender computer device accessing the sender account to generate on a display device of a sender computer device, a message category input graphical user interface (GUI) element configured to receive a message category input limited to a member of the permitted message category set, for association with a message addressed to the receiver account;
(c) receiving from the sender computer device, and storing in the database, the message category input in association with the message addressed to the receiver account; and
(d) communicating with a receiver computer device accessing the receiver account to generate on a display device of the receiver computer device:
   (i) for each member of the permitted message category set, a message category GUI element corresponding to a different one of the members of the permitted message category set; and
   (ii) a message indicator GUI element indicative of the message addressed to the receiver account stored in the database, in association with the message category GUI element corresponding to the message category input.

In an embodiment of the method, the method further comprises the step of: storing in the database in association with the receiver account, for each member of the permitted message category set, an associated permitted sender client account set. The message category input GUI element is configured to further limit the message category input to a member of the permitted message category set for which the sender account is a member of the associated permitted sender client account set.

In an embodiment of the method, the permitted message category set or the permitted sender client account set, or both, may be configurable from a computer device accessing the receiver account.

In an embodiment of the method, the method further comprises the steps of:
(a) storing in the database in association with the receiver account:
   (i) an automatic-forwarding message category set; and
   (ii) for each member of the automatic-forwarding message category set, an associated automatic-forwarding client account set;
(b) determining if a condition is satisfied, the condition comprising at least that the message category input corresponds to a member of the automatic-forwarding message category set;
(c) when the condition is satisfied, generating and storing in the database, a forwarding message comprising at least a portion of the message addressed to the receiver account, and addressed to each member of the automatic-forwarding client account set associated with the member of the automatic-forwarding message category set; and
(d) communicating with a client computer device accessing a member of the automatic-forwarding client account set to generate on a display device of the client computer device, the forwarding message.

In an embodiment of the method, the method further comprises the steps of:
(a) storing in the database in association with the receiver account, an automatic-reply message category set; and
(b) determining if a condition is satisfied, the condition comprising at least that the message category input corresponds to a member of the automatic-reply message category set;
(c) when the condition is satisfied, generating and storing in the database, a replying message addressed to the sender account; and
(d) communicating with a client computer device accessing the sender account to generate on a display device of the client computer device, the replying message.

In an embodiment of the above method, the method further comprises the step of storing in the database in association with the receiver account, for each member of the automatic-reply message category set, an associated reply message template. The replying message comprises the reply message template associated with the member of the automatic-reply message category set.

In an embodiment of the method, each message category GUI element comprises a spatially distinct region on the display device of the receiver computer device. The message indicator GUI element is associated with the message category GUI element corresponding to the message category input by being disposed within the region corresponding to the message category input.

In an embodiment of the method, each message category GUI element comprises an item in a selectable list. The message indicator GUI element is associated with the message category GUI element corresponding to the message category input by being displayed when the item corresponding to the message category input is selected from the list. The message indicator GUI element may comprise one or a combination of an identifier for the sender account, a message subject line, or a date or time corresponding approximately to when the message addressed to the receiver account was stored in the database.

In an embodiment of the method, the communication network comprises the Internet.

In another aspect, the present invention comprises a system for electronic messaging between client accounts including a sender account and a receiver account. The system comprises: a server computer device; a communications interface operatively connected to the server computer device for communicating via a communication network with computer devices; a computer-readable database operatively connected to the server computer device, and storing in association with the receiver account, a permitted message category set; and a non-transitory computer readable medium operatively connected to the server computer device, and storing instructions executable by the server computer device to implement a method. The method comprises the steps of:
(a) communicating with a sender computer device accessing the sender account to generate on a display device of a sender computer device, a message category input graphical user interface (GUI) element configured to receive a message category input limited to a member of the permitted message category set, for association with a message addressed to the receiver account;
(b) receiving from the sender computer device, and storing in the database, the message category input in association with the message addressed to the receiver account; and
(c) communicating with a receiver computer device accessing the receiver account to generate on a display device of the receiver computer device:
   (i) for each member of the permitted message category set, a message category GUI element corresponding to a different one of the members of the permitted message category set; and
   (ii) a message indicator GUI element indicative of the message addressed to the receiver account stored in the database, in association with the message category GUI element corresponding to the message category input.

In an embodiment of the system, the database is storing in association with the receiver account, for each member of the permitted message category set, an associated permitted sender client account set. The message category input GUI element is configured to further limit the message category input to a member of the permitted message category set for which the sender account is a member of the associated permitted sender client account set.

In an embodiment of the system, the permitted message category set or the permitted sender client account set, or both may be configurable from a computer device accessing the receiver account.

In an embodiment of the system, the database is storing in association with the receiver account: an automatic-forwarding message category set; and for each member of the automatic-forwarding message category set, an associated automatic-forwarding client account set. The method further comprises the steps of:
(a) determining if a condition is satisfied, the condition comprising at least that the message category input corresponds to a member of the automatic-forwarding message category set;
(b) when the condition is satisfied, generating and storing in the database, a forwarding message comprising at least a portion of the message addressed to the receiver account, and addressed to each member of the automatic-forwarding client account set associated with the member of the automatic-forwarding message category set; and
(c) communicating with a client computer device accessing a member of the automatic-forwarding client account set to generate on a display device of the client computer device, the forwarding message.

In an embodiment of the system, the database is storing in association with the receiver account, an automatic-reply message category set. The method further comprises the steps of:
(a) determining if a condition is satisfied, the condition comprising at least that the message category input corresponds to a member of the automatic-reply message category set;
(b) when the condition is satisfied, generating and storing in the database, a replying message addressed to the sender account; and
(c) communicating with a client computer device accessing the sender account to generate on a display device of the client computer device, the replying message.

In an embodiment of the above system, the database is storing in association with the receiver account, for each member of the automatic-reply message category set, an associated reply message template. The replying message comprises the reply message template associated with the member of the automatic-reply message category set.

In an embodiment of the system, each message category GUI element comprises a spatially distinct region on the display device of the receiver computer device. The message indicator GUI element is associated with the message category GUI element corresponding to the message category input by being disposed within the region corresponding to the message category input.

In an embodiment of the system, each message category GUI element comprises an item in a selectable list. The message indicator GUI element is associated with the message category GUI element corresponding to the message category input by being displayed when the item corresponding to the message category input is selected from the list. The message indicator GUI element may comprise one or a combination of an identifier for the sender account, a message subject line, or a date or time corresponding approximately to when the message addressed to the receiver account was stored in the database.

In an embodiment of the system, the communication network comprises the Internet, and the system comprises a web server.

In another aspect, the present invention comprises a program product comprising a non-transitory computer readable medium storing instructions executable by a server computer device operatively connected to a computer-readable database storing in association with the receiver account a permitted message category set, and to a communications interface for communicating via a communications network with computer devices, to implement a method for electronic messaging between client accounts including a sender account and a receiver account. The method comprises the steps of:
(a) communicating with a sender computer device accessing the sender account to generate on a display device of a sender computer device, a message category input graphical user interface (GUI) element configured to receive a message category input limited to a member of the permitted message category set, for association with a message addressed to the receiver account;
(b) receiving from the sender computer device, and storing in the database, the message category input in association with the message addressed to the receiver account; and
(c) communicating with a receiver computer device accessing the receiver account to generate on a display device of the receiver computer device:
   (i) for each member of the permitted message category set, a message category GUI element corresponding to a different one of the members of the permitted message category set; and
   (ii) a message indicator GUI element indicative of the message addressed to the receiver account stored in the database, in association with the message category GUI element corresponding to the message category input.

In an embodiment of the program product, the database is storing in association with the receiver account, for each member of the permitted message category set, an associated permitted sender client account set. The message category input graphical user interface (GUI) element is configured to further limit the message category input to a member of the permitted message category set for which the sender account is a member of the associated permitted sender client account set.

In an embodiment of the program product, the permitted message category set or the permitted sender client account set, or both may be configurable from a computer device accessing the receiver account.

In an embodiment of the program product, the database is storing in association with the receiver account: an automatic-forwarding message category set; and for each member of the automatic-forwarding message category set, an associated automatic-forwarding client account set. The method further comprises the steps of:
(a) determining if a condition is satisfied, the condition comprising at least that the message category input corresponds to a member of the automatic-forwarding message category set;
(b) when the condition is satisfied, generating and storing in the database, a forwarding message comprising at least a portion of the message addressed to the receiver account, and addressed to each member of the automatic-forwarding client account set associated with the member of the automatic-forwarding message category set; and
(c) communicating with a client computer device accessing a member of the automatic-forwarding client account set to generate on a display device of the client computer device, the forwarding message.

In an embodiment of the program product, the database is storing in association with the receiver account, an automatic-reply message category set. The method further comprises the steps of:
(a) determining if a condition is satisfied, the condition comprising at least that the message category input corresponds to a member of the automatic-reply message category set;
(b) when the condition is satisfied, generating and storing in the database, a replying message addressed to the sender account; and
(c) communicating with a client computer device accessing the sender account to generate on a display device of the client computer device, the replying message.

In the an embodiment of the above program product, the database is storing in association with the receiver account, for each member of the automatic-reply message category set, an associated reply message template. The replying message comprises the reply message template associated with the member of the automatic-reply message category set.

In another aspect, the present invention comprises a method for generating an electronic message from a sender account addressed to a receiver account. The method is implemented using a sender computer device comprising a display device and in communication via a communication network with a database storing in association with the receiver account, a permitted category message set. The method comprises the steps of:
(a) generating on the display device, a message category input graphical user interface (GUI) element configured to receive a message category input limited to a member of the permitted message category set stored in the database, for association with a message addressed to the receiver account;
(b) using the GUI element to provide a message category input for association with the message addressed to the receiver account; and
(c) initiating transmission to the database for storage, the message category input in association with the message addressed to the receiver account.

In another aspect, the present invention comprises a method for handling an electronic message from a sender account addressed to a receiver account. The method is implemented using a receiver computer device comprising a display device and in communication via a communication network with a database storing the message in association with a message category input limited to a member of a permitted category set stored in the database in association with the receiver account. The method comprises the steps of:
(a) for each member of the permitted message category set, a message category GUI element corresponding to a different one of the members of the permitted message category set; and
(b) generating on the display device, a message indicator GUI element indicative of the message addressed to the receiver account stored in the database, in association with the message category GUI element corresponding to the message category input.

### BRIEF DESCRIPTION OF THE DRAWINGS

In the drawings shown in the specification, like elements may be assigned like reference numerals. The drawings are not necessarily to scale, with the emphasis instead placed upon the principles of the present invention. Additionally, each of the embodiments depicted are but one of a number of possible arrangements utilizing the fundamental concepts of the present invention.

FIG. 1 is a schematic illustration of an embodiment of a system of the present invention in communication via the Internet with multiple client computer devices, used by different clients in a health care organization.

FIG. 2 is a flow chart of an embodiment of a method of the present invention for sending messages from a client account addressed to another client account.

FIG. 3 is a flow chart of an embodiment of the method of the present invention for automatically forwarding a message received from a client account to another client account.

FIG. 4 is a flow chart of an embodiment of the method of the present invention for automatically replying to a message received from a client account.

FIG. 5 is an embodiment of a GUI, generated on a display device of a client computing device, for logging into an existing client account, or for creating a new client account, in accordance with the present invention.

FIG. 6 is an embodiment of a GUI, generated on a display device of a client computing device, for choosing a new client account type based on a client's role, in accordance with the present invention.

FIG. 7 is an embodiment of a GUI, generated on a display device of a client computing device, for a client to enter information for creating a new client account, in accordance with the present invention.

FIG. 8 is an embodiment of a GUI, generated on a display device of a client computing device logged into a client account, for selecting from one of several available applications including a secure message application, in accordance with the present invention.

FIG. 9 is an embodiment of a GUI, generated on a display device of a client computing device logged into a client account, showing a dashboard view of a secure message application, in accordance with the present invention.

FIG. 10 is an embodiment of a GUI, generated on a display device of a client computing device logged into a client account, for generating a new message to another account, in accordance with the present invention.

FIG. 11 is an embodiment of a GUI, generated on a display device of a client computing device logged into a client account, showing a list view of indicators of received messages in multiple message categories, in accordance with the present invention.

FIG. 12 is an embodiment of a GUI, generated on a display device of a client computing device logged into a client account, showing a timeline view of indicators of received messages multiple message categories, in accordance with the present invention.

FIG. 13 is an embodiment of a GUI, generated on a display device of a client computing device logged into a client account, showing input GUI elements for prescribing rules for automatically replying to messages received by the client account.

### DETAILED DESCRIPTION OF PREFERRED EMBODIMENT

Definitions and interpretation. Any term or expression not expressly defined herein shall have its commonly accepted definition understood by a person skilled in the art. As used herein, the following terms have the following meanings.

“Computer device” and like terms refer to one or more electronic devices capable of performing operations on data. Non-limiting examples of computer devices include devices referred commonly referred to as processors, servers, general purpose computers, personal computers, desktop computers, laptop computers, handheld computers, smart phones, tablet computers, and the like. Any kind of computer device adapted for carrying out the methods described herein may be used.

“Display device” in relation to a computer device, refers to any electronic device capable of presenting information in visual form. Non-limiting examples of display devices include electronic monitors and display panels regardless of their underlying technology (e.g., CRT, LED, LCD, PDP, and the like).

“Computer readable medium” or “CRM” refers to a non-transitory, tangible medium capable of persistently encoding information in a format readable by a computer device. Non-limiting examples of CRMs include magnetic media (e.g., a magnetic diskette or tape), optical media (e.g., an optical disc), and solid-state media using integrated circuits (e.g., flash memory).

“Computer program product” refers to a computer-readable medium storing a set of instructions in any language, code or notation, that causes a computer device to perform a particular function, whether directly or indirect after conversion to another language, code or notation.

“Communication network” refers to a network enabling electronic communications between computer devices. Non-limiting examples of communication networks include one or a combination of the Internet, a local area network or organization intranet, and a telephone network, whether wired or wireless.

The use of the terms “computer device”, “computer readable medium”, “computer program product” and “communication network” in the singular, and depiction of such elements as a single physically discrete element include the possibility of such computer elements comprising multiple physically discrete devices operatively connected to each other. Accordingly, the present invention may be implemented in a centralized fashion by a single physically discrete computer element, or in a distributed fashion by multiple physically discrete computer elements, which are operatively connected to each other, and which may be remotely situated from each other.

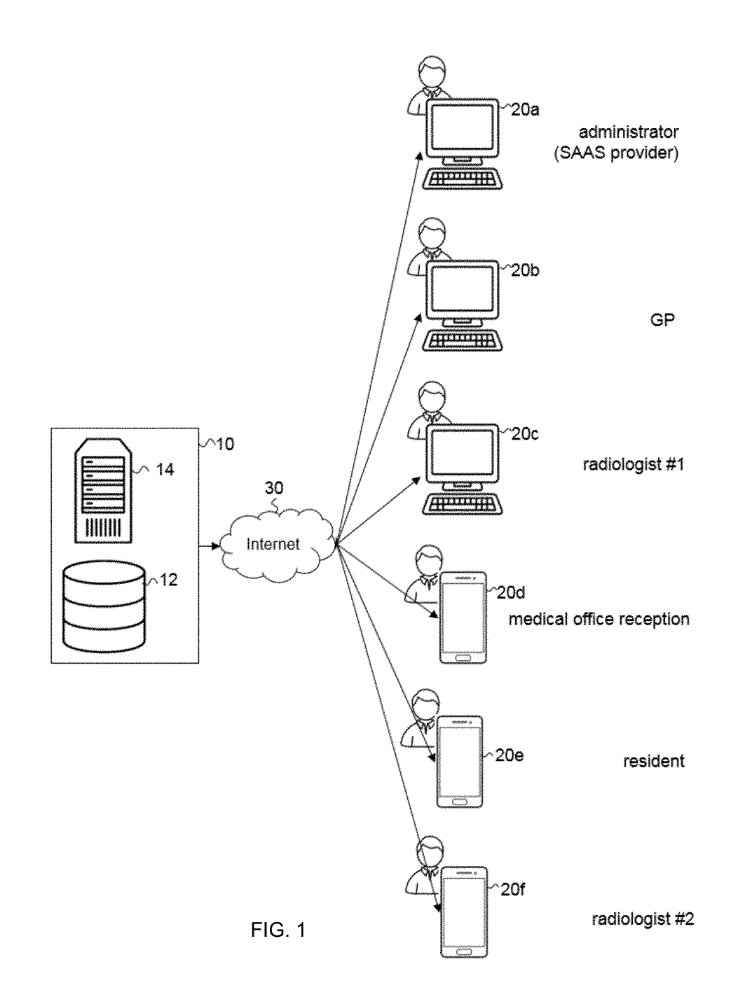

System and Computer Program Product. FIG. 1 is a schematic illustration of an embodiment of a system (10) of the present invention interacting with multiple client computer devices (20) via a communication network (30).

The system (10) includes a database (12) operatively connected to a server computer device operatively connected to a non-transitory CRM and a communication interface for communicating with the client computer devices (20) via a communication network (30). The server computer device, CRM and communication interface are collectively represented by icon (14). The database (12) stores information client profile information pertaining to client accounts, and messages addressed to client accounts (as will be discussed below). In the embodiment of FIG. 1, the communication network (30) comprises the Internet, and the system (10) comprises a web server that processes requests received via the Internet from the client computer devices (20). The server computer device (14) executes instructions stored on the CRM to implement an embodiment of a method of the present invention. Accordingly, the CRM storing instructions that enable the server computer device to implement an embodiment of a method of the present invention constitutes a program product of the present invention.

The client computer devices (20) are used by human clients to interact with the system (10). Each of the client computer devices (20) includes a display device. The system (10) is not limited by any specific number of client computer devices (20) or type of client computer devices (20) that may interact with the system (10) at any given time. In practice, for example, the system (10) may interact with tens of thousands of client computer devices (20) accessing an equivalent number of client accounts at a given time. As a non-limiting illustrative example, FIG. 1 shows the system (10) interacting with six client computer devices (20a to 20f). Client computer devices (20a, 20b, 20c) are desktop computer workstations, each of which is configured to run a generic web browser for retrieving and presenting web pages via the World Wide Web for interacting with the system (10). Client computer devices (20d, 20e, 20f) are mobile smart phones or tablet computers, each of which is configured to run a dedicated mobile application for interacting with the system (10).

The present invention is not limited by any particular industrial application. For the purpose of a non-limiting illustrative example, FIG. 1 shows the system being used in a health care context by human clients notionally identified as follows:
“administrator” is a cloud-based “software as a service” (SAAS) provider that oversees the system (10) for use by a health care organization via computer device (20a);
“GP”, “radiologist #1”, “resident”, and “radiologist #2” are physicians using the system (10) to send messages to and receive messages from other client accounts, via computer devices (20b, c, e, and f, respectively); and
“medical office reception” is a non-physician staff member of radiologist #1 using the system (10) to send messages to and receive messages from other client accounts, via computer device (20d).

Although FIG. 1 shows different clients interacting with the system (10) using different computer devices (20), more than one client may use the same computer device (20) to access their respective client accounts, and a single client may use multiple computer device (20) to access his or her client account.

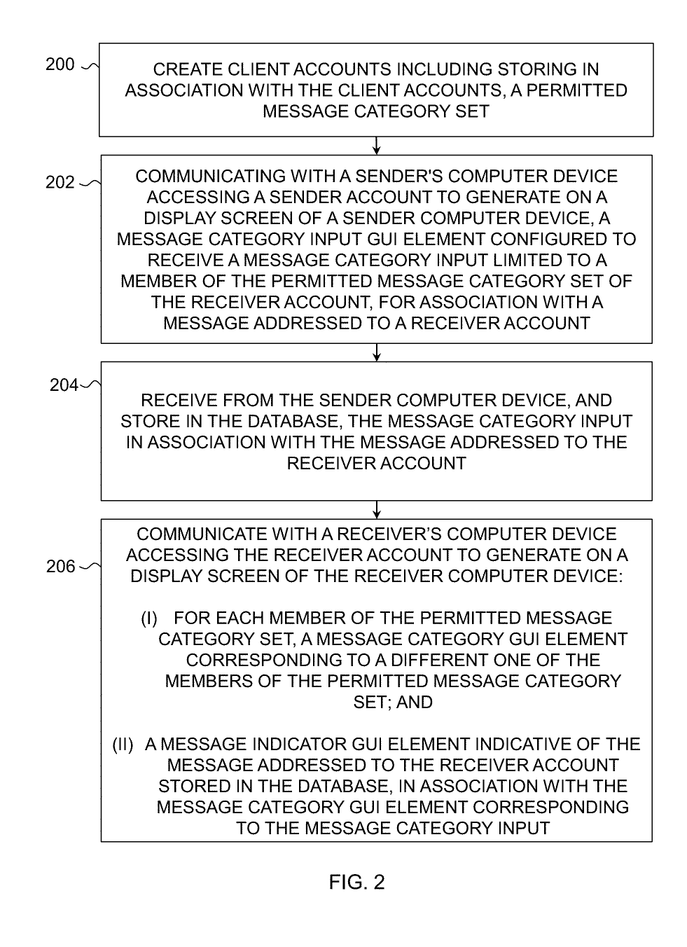

Method for sending and receiving categorized messages. FIG. 2 is a flow chart of an embodiment of a method of the present invention, which is described below.

Client Account Creation. The embodiment of the method begins with the creation and storage of multiple client accounts in the database (12) (FIG. 2, step 200). Different client accounts are uniquely associated with different human clients to define different authorizations with respect to the system (10). The present invention is not limited by any particular way in which the client accounts are created in the database (12).

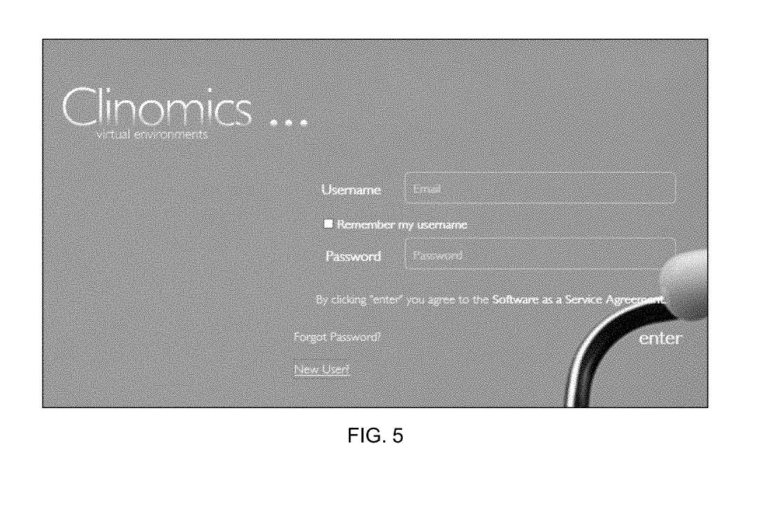

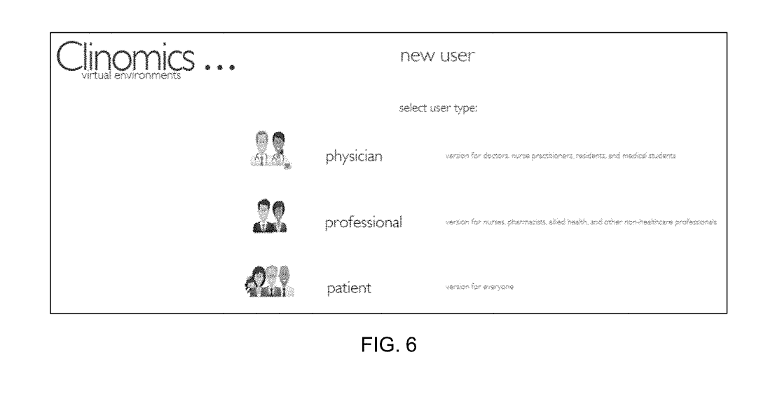

Continuing with the illustrative example, clients (other than the administrator) initiate the client account creation process using a web browser on a client device (20) to access a “Login” web page (FIG. 5) and selecting the link labelled “New User?”, which directs the web browser to the “New User” web page (FIG. 6). The “New User” web page presents three selectable options for different types of client accounts depending on the client's role:
“physician” for clients who are doctors, nurse practitioners, residents and medical students;
“professional” for clients who are nurses, pharmacists, allied health and non-health care professionals“; and
“patient” for all other types of clients.

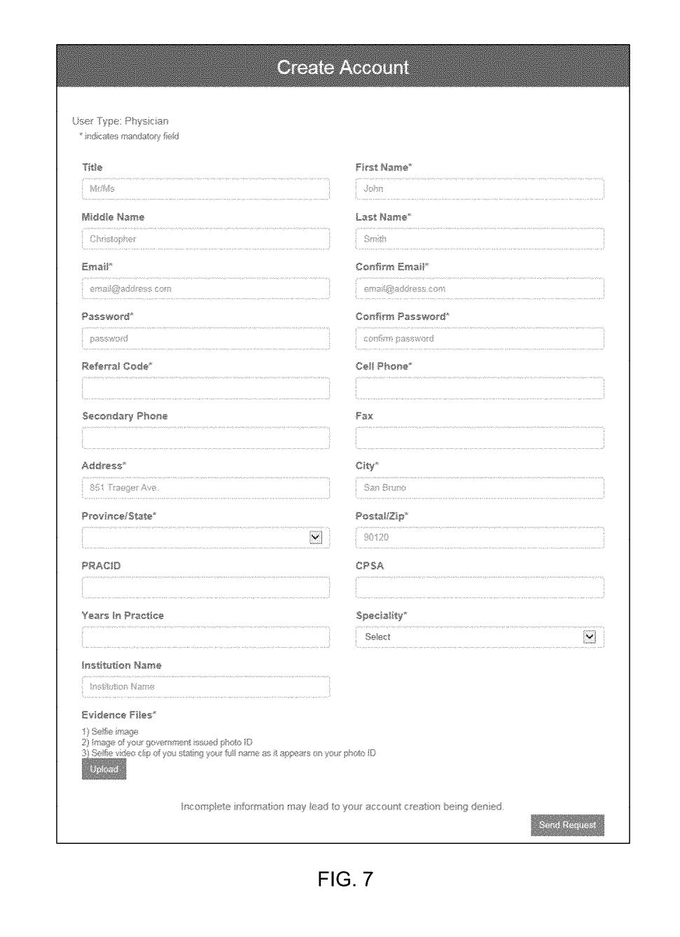

In the illustrative example, the GP, radiologist #1, resident, and radiologist #2 select the link for the option “physician” (FIG. 6). This directs the web browser to the “Create Account” web page (FIG. 7) having input fields that apply to physicians. The “Create Account” web page contains a number of text input fields for client identification and authentication, and requests the client to upload a photograph of himself or herself, an image of government issued photo identification, and a video clip of himself or herself stating his or her name as it appears on the photo identification. Once the potential client has supplied all the necessary information, the potential client selects the “Send Request” button (FIG. 7). This initiates uploading of the new client information to the database (12) where it is stored. The administrator may then review the information and approve the creation of a new client account. The medical office assistant and patients create accounts in a similar manner except that he or she selects the link for the option “professional” and “patient”, respectively, (FIG. 6), which would direct the web browser to a “Create Account” web page (not shown) similar to that of FIG. 7, but having input fields that apply to non-physician professionals or patients, as the case may be.

Permitted message category set. The embodiment of the method involves storing a ““permitted message category set”” in association with each of the client accounts (FIG. 2, step 200). The “permitted message category set” is a collection of one or more message categories under which a particular client account can receive messages, and hence under which other client accounts are permitted to send messages addressed to the receiver account. The present invention is not limited by any particular way in which the “permitted message category sets” are defined and stored in association with client accounts in the database (12).

Continuing with the illustrative example, in one embodiment, the administrator defines a default “permitted message category set” for client accounts created under the “physician”, “professional” or “patient” role. For example, the default message category set for client accounts created under the “physician” consists of the following message categories: “on-call consults”, “E-consultation”, “new referrals”, “from physician/clinics”, “from consultant”, “group message”, “from my office”, “from allied health”, “cool case”, and “social call”. The client accounts created under the “professional role” consists of only the message category “from office”.

In embodiments, the system (10) may allow the administrator and/or the client to customize the default “permitted message category set” associated with the client account by the addition or subtraction of a message category. The system may also allow the administrator and/or the client to customize the client accounts that are permitted to send messages to that client account, in that message category, either by the role(s) of the client account or by individual identifier of the client account(s) sending the message.

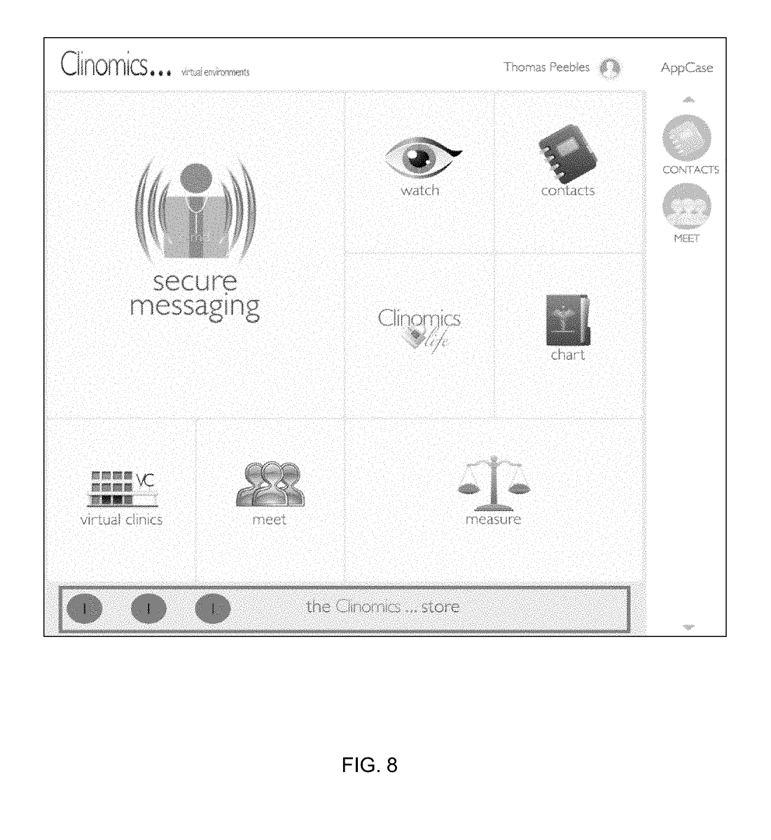

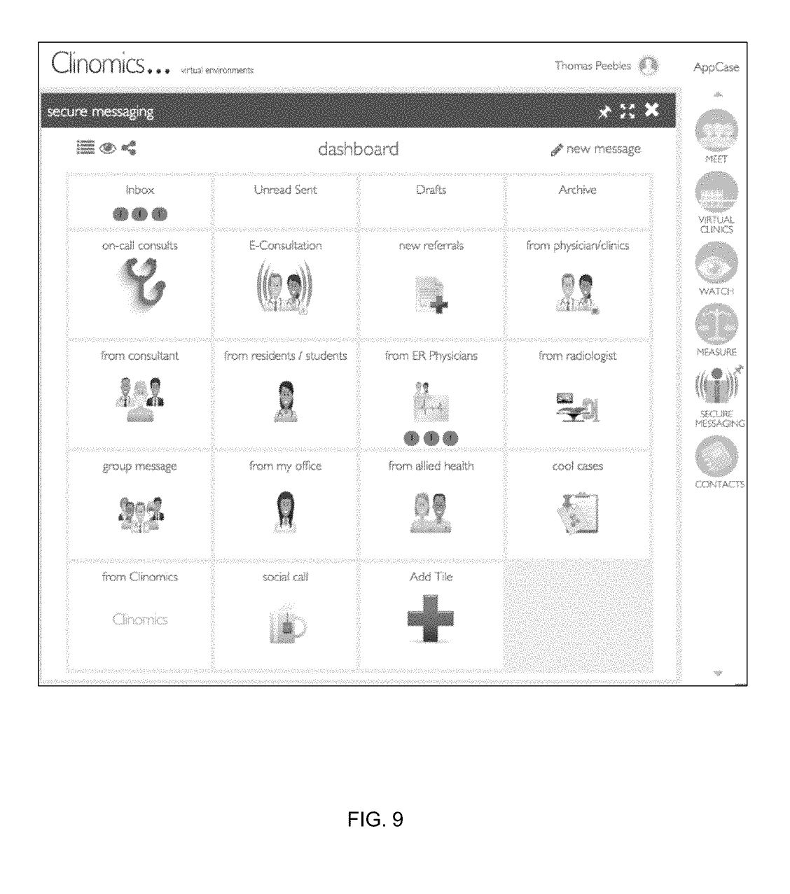

Continuing with the illustrative example, for instance, the previously receiver account holder uses a web browser on a client computer device (20) to access the “Login” web page (FIG. 5). The receiver account holder enters his or her email address in the “Username” field, and a character string password in the “Password” field (as specified during the client account creation process), and clicks on the “enter” button, to access the system (10). For convenient discussion, the client account which has been logged into is now referred to as the “receiver account”. The system (10) directs the web browser to a “landing page” web page (FIG. 8) with a variety of applications in different tiles. The client clicks on the tile labelled “secure messaging” to access a secure messaging application. The system (10) directs the web browser to a “dashboard” web page (FIG. 9).

The dashboard web page (FIG. 9) displays a variety of tiles. Each of the tiles corresponds to member of the “permitted message category set” stored in association with the receiver account. Some of the tiles may correspond to members of the default “permitted message category set” as defined by the administrator. For example, the tiles “E-consultation” and “new referrals” may be members of the default “permitted message category set” defined by the administrator for all client accounts created under the “physician” role. Some of the tiles may correspond to members of the “permitted message category set” that have been added in a customized manner for the particular client account. For example, the tiles “from residents/students” and “from radiologist” may have been added to the “permitted message category set” of the GP account by the GP clicking on the “Add Tiles” tile. This activates a user facility with a GUI whereby the GP can create and add “from residents/students” and “from radiologist” as customized members of the “permitted message category set”. Further, the user facility may allow the GP to select specific graphical icons for the tiles corresponding to these message categories, such as the x-ray machine icon shown in the “from radiologist” tile. Accordingly, the dashboard web page can be highly customized to the needs of each client account.

Moreover, the user facility may allow the GP to specify, for each member of the “permitted message category set”, an associated “permitted sender client account set”. For each member of the “permitted message category set”, the associated “permitted sender client account set” is a collection of one or more client accounts that are permitted to send messages to the receiver account in association with that associated message category. For example, the GP may specify that the “permitted sender client account set” for the “from radiologist” message category consists of the radiologist #1 and radiologist #2 accounts only. Thus, the radiologist #1, radiologist #2 accounts would be permitted to send messages associated with the “from radiologist” category, but the resident account and the medical office reception account would not be permitted to do so. In contrast, the GP may specify that the “permitted sender client account set” for the “cool cases” message category includes any client account created under the “physician” role. Thus, the radiologist #1, radiologist #2, and resident accounts created under the “physician” role would be permitted to send messages associated with the “cool cases” category, but the medical office reception account created under the “professional” role would not be permitted to do so. Accordingly, the receiver account can be highly customized to control which sender accounts can send messages to the receiver account, and the message categories under which the messages can be sent.

Graphical user interface for sender account. The embodiment of the method continues with communicating with a sender computer device accessing a sender account to generate on a display device of a sender computer device, a “message category input graphical user interface (GUI) element” configured to receive a “message category input” limited to a member of the “permitted message category set” of the receiver account, for association with a message addressed to the receiver account (FIG. 2, step 202). In this context, the “sender account” refers to a client account from which the message is being sent, and the “receiver account” refers to the client account to which the message is addressed.

The “message category input GUI element” may be any GUI input element that allows a client accessing the sender account to select one of the members of the “permitted message category set” stored in association with that receiver account, while preventing that client from selecting a message category that is not a member of that “permitted message category set”. The person skilled in the art will be able to implement the “message category input GUI element” using GUI elements known in the art. Non-limiting suitable examples of suitable GUI elements may include one or a combination of text fields, checkboxes, radio buttons, dropdown lists, list boxes, toggles, pickers, and the like, as well as “error handling” routines with alerts or messages that prevent a client from selecting a message category that is not a member of a “permitted message category set” stored in association with the receiver account.

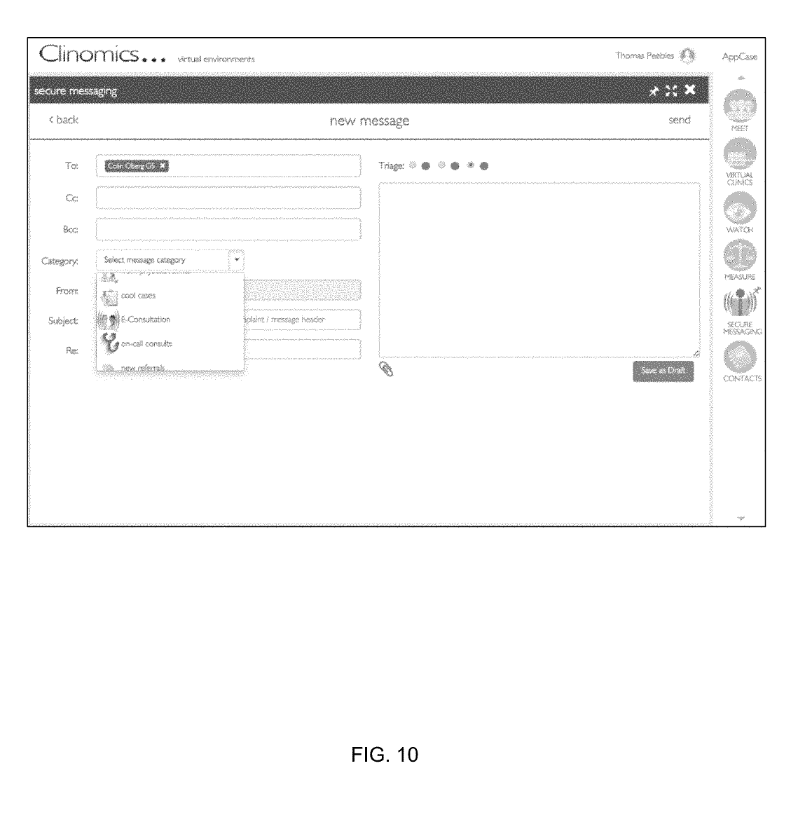

Continuing with the illustrative example, the holder of the sender account would log into the system under the “sender account” and proceed to the “dashboard” web page (FIG. 9) in the same manner as described for the receiver account holder. Once the sender account holder has reached the dashboard webpage (FIG. 9), he or she clicks on the link labelled “new message”, which directs the web browser to a “new message” web page (FIG. 10). The “new message” web page has a variety of GUI input elements for entering message information as follows:
“To”, “Cc” and “Bcc” text input fields for entering the identifier of the client account to which the message is addressed;
“From” text field, which is automatically completed with the name of the logged in sender account from which the message is being sent;
“Subject” text input field for entering a brief description of the message;
“Re” text input field for entering a patient name and date of birth;
text input field (right hand side of FIG. 10) for entry of a message body;
a paper clip icon, which can be selected to initiate a utility for file attachment; and
“Triage” radio buttons for selecting a color-coded priority indicator for the message, with the red button signifying an emergent/high priority message, a yellow button signifying an urgent/mid priority message, and a green button message signifying an elective/low priority message.

The illustrative example of FIG. 10 also includes the “message category input GUI element” in the form of a dropdown list labelled “Category”. Upon being selected, the dropdown list displays one or more items limited to the member(s) of the “permitted message category set” stored in association with receiver account, having regard to any restrictions imposed by associated “permitted sender client account sets. Thus, in one embodiment, the list of items available for selection in dropdown list may be dynamically updated depending on the name of the receiver account entered in the “To” field, as well as the identity of the sender account logged into the system. For example, suppose that GP account is the receiving account. It will be recalled that the GP account “permitted message category set” has been customized to include the “from radiologist” message category, and the “permitted sender client account set” associated with that message category consists of only the radiologist #1 and radiologist #2 accounts. Further, it will be recalled that the GP account's “permitted message category set” has been customized to include the “cool cases” message category, and that the “permitted sender client account set” associated with that message category consists of any client account created under the “physician” role. Accordingly, if the sender account is the “radiologist #1 account”, and the GP account is entered into the “To” field, then the dropdown list will show the message categories “from radiologist” and “cool cases” available for selection. In contrast, if the sender account is the “resident account”, and the GP account is entered into the “To” field, then the dropdown list will show the message category “cool cases” available for selection, but not the “from radiologist” message category. Now suppose that the sender account is the resident account, and the GP account is entered into the “To” field and the radiologist #1 account is entered into the “Cc” field. In these circumstances, the drop down list will be limited to only the message category(s) that are members of both the permitted message category set for the GP account and the permitted message category set for the radiologist #1 account—that is to say, the intersection of both permitted message category sets. For example, that intersection may include a message category called “group message”, which is included in the default “permitted message category set” defined by the administrator to address the circumstances where there is no other common message category amongst the GP account and the radiologist #1 account.

Storing the message. The embodiment of the method continues with receiving from the sender computer device, and storing in the database, the “message category input” in association with the message addressed to the receiver account (FIG. 2, step 204).

Continuing with the illustrative example, once the message has been created using the “new message” web page (FIG. 10), the client clicks on the “send” button to initiate uploading of the entered message information to the database (12), so that it can be later retrieved by the receiver account to which the message is addressed.

Graphical user interface for receiver account. The embodiment of the method continues with communicating with a receiver computer device accessing the receiver account to generate on a display device of the receiver computer device: for each member of the permitted message category set, a “message category GUI element” corresponding to a different one of the members of the “permitted message category set”; and a “message indicator GUI element” indicative of the message addressed to the receiver account stored in the database, in association with the “message category GUI element” corresponding to the “message category input” (FIG. 2, step 206). A purpose of the message category GUI elements and the message indicator GUI element is to assist the client accessing the receiver account to ascertain the receipt of the message, and its associated “message category input”.

Continuing with the illustrative example, a client may uses a web browser on a client device (20) to log into the “receiver account” and proceed to the “dashboard” web page (FIG. 9). In the dashboard web page, the “message category GUI elements” are in the form of spatially distinct tiles, each of which is labelled with a different one of the message categories of the “permitted message category set” stored in association with the receiver account. The “message indicator GUI elements” are disposed within the tiles, and indicate the count of unread messages addressed to the receiver account that are stored in the database (12) in association with a particular message category. Different message indicators are used to indicate the count of messages having different priority levels, as selected using the “Triage” radio buttons during the message creation process as described above. For example, the dashboard web page of FIG. 9 shows the digit “1” superimposed on each one of a red circle, a yellow circle, and a green circle within the tile labelled “from ER Physicians”, to signify that the database (12) stores three unread messages addressed to the receiver account in association with the message category “from ER Physicians”, which are associated with a high priority, mid priority and low priority status, respectively.

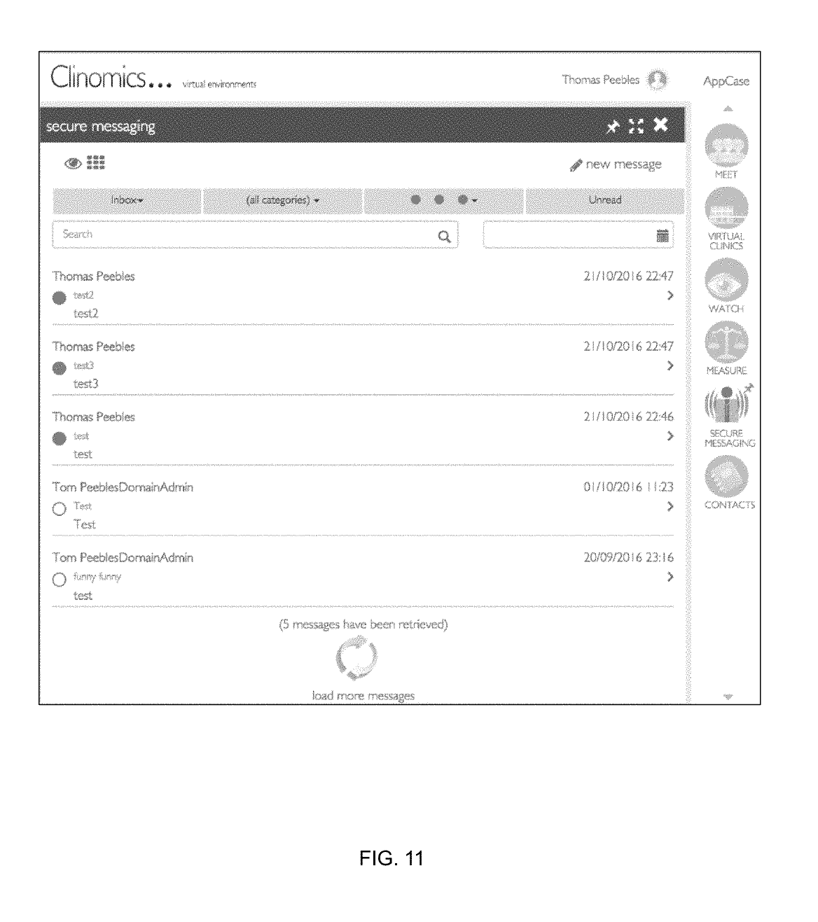

In the illustrative example, the client can also use the web browser to view a web page providing a list view of the messages (FIG. 11), in which the “message category GUI elements” are in the form of a dropdown list of selectable items, each of which represents a different one of the message categories of the “permitted message category set” stored in association with the receiver account. The “message indicator GUI elements” are in the form of header information for the messages: i.e., an identifier for the sender account, a message subject line, or a date or time corresponding approximately to when the message addressed to the receiver account was stored in the database (12). In FIG. 11, the message category selected in the dropdown list is labelled “(all categories)”, which corresponds to a list view of received messages in members of the “permitted message category set” stored in association with the receiver account. Alternatively, a single one of the other message categories can be selected from the dropdown list to show header information only for messages stored in association with a particular member of the “permitted message category set” stored in association with the receiver account.

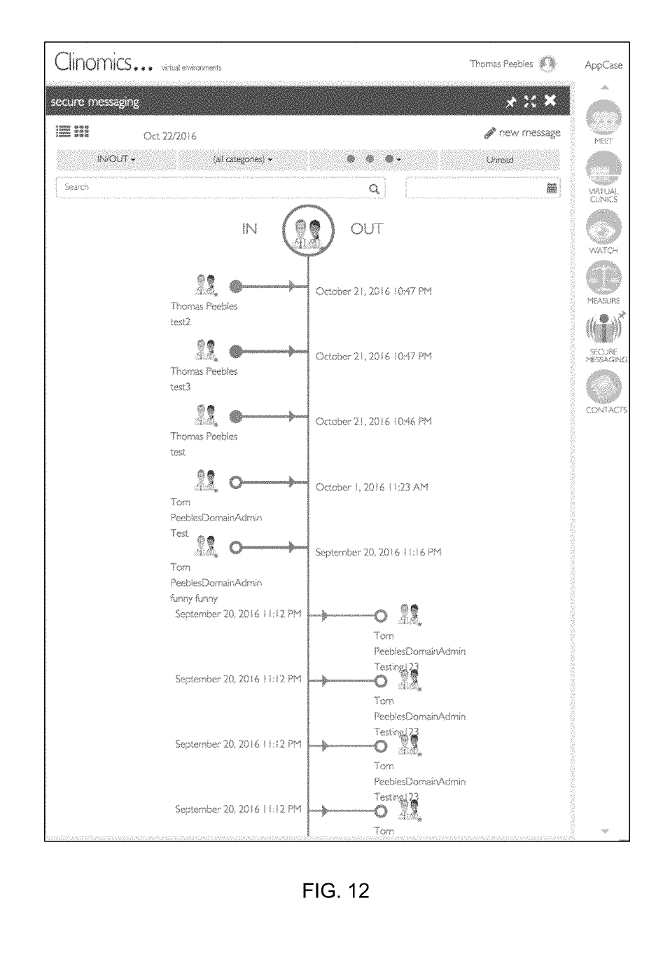

In the illustrative example, the client can also use the web browser to view a web page providing a timeline view of the messages (FIG. 12). The timeline view is similar to the list view (FIG. 11), except that the message indicator GUI elements are arranged in chronological order along a vertically extending timeline. The timeline is associated with the client account that is logged into the system. Arrow-headed lines indicate the flow of messages to and from the logged-in client account. Arrow-headed lines pointing towards the timeline are indicative of messages received by the logged-in client account, while arrow-headed lines pointing away from the timeline are indicative of messages sent from the logged-in client account. Received incoming messages are positioned on the left-hand side of the timeline, while sent outgoing messages are positioned on the right-hand side of the timeline. Each arrow-headed line is associated with a circular marker. The marker is shaded solid if the message has been displayed for reading by the client account to which the message is addressed, and shown as empty if the message has not yet been displayed by reading by the client account to which the message is addressed.

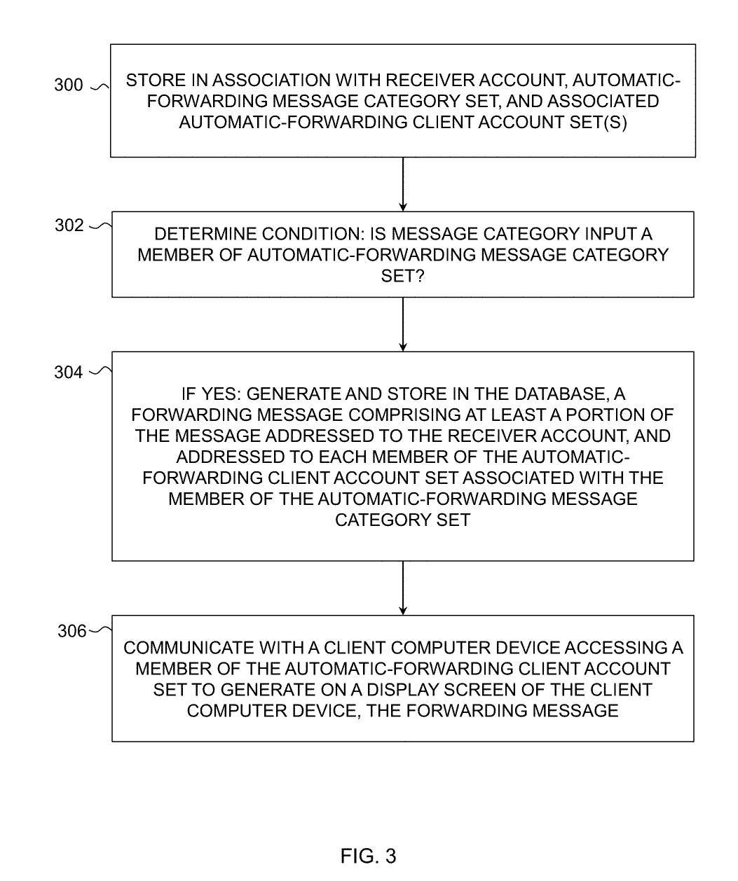

Method for automatic forwarding of categorized messages. FIG. 3 shows optional steps for automatically forwarding a message received by the receiver account. These steps allow messages from different categories to be selectively forwarded (or not forwarded) to potentially different client accounts, with no involvement of the client receiving the message beyond setting automatic forwarding rules.

The auto-forwarding method involves storing in the database in association with the receiver account: an “automatic-forwarding message category set”; and for each member of the automatic-forwarding message category set, an associated “automatic-forwarding client account set” (FIG. 3, step 300). The “automatic-forwarding message category set” is a collection of one or more message categories for received messages that are to be automatically forwarded to other client accounts. The “automatic-forwarding client account set” is a collection of one or more client accounts to which a message is to be forwarded.

Continuing with the illustrative example, suppose for instance that the “automatic-forwarding message category set” for the radiologist #1 account consists of the members “E-consultation” and “cool cases”. For the “E-consultation” message category, suppose that the associated “automatic-forwarding client account set” consists of the medical office reception account. For the “cool cases” message category, suppose that the associated automatic-forwarding client account set consists of the radiologist #2 account.

The auto-forwarding method next involves determining if the message addressed to the receiver account satisfies a condition is satisfied (FIG. 3, step 302). Minimally, the condition includes that the “message category input” of the message is a member of the “automatic-forwarding message category set”. The condition may include other requirements such as the message being received within a specified date range and/or time range.

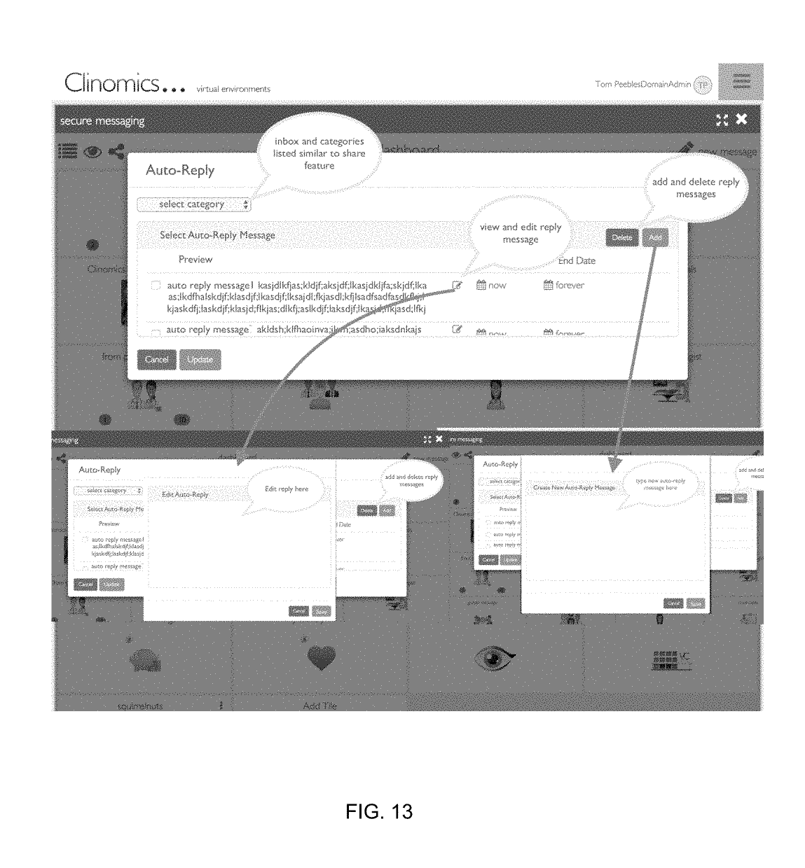

In embodiments, a GUI may be provided for the client to configure an “automatic-forwarding message category set”, and an associated “automatic-forwarding client account set” and condition. The GUI may be similar to that shown in FIG. 13 in respect to the automatic-reply method of the present invention, discussed below.

Continuing with the illustrative example, suppose that three messages addressed to the radiologist #1 account are stored in the database (12), as follows:
Message #1 is received from the GP account with the “message category input” “E-consultation”, and requests a patient consultation. Message #1 satisfies the condition of being within automatic-forwarding message category set (as defined above), and will therefore be automatically forwarded;
Message #2 is received from the resident account with the “message category input” “cool case”, and attaches a journal article describing a medical case of interest to radiologists. Message #2 satisfies the condition of being within automatic-forwarding message category set (as defined above), and will therefore be automatically forwarded; and
Message #3 is received from the medical office reception account with the “message category input” “from office”, and pertains to office administration. Message #3 is not a member of the automatic-forwarding message category set (as defined above) and therefore will not be automatically forwarded.

When the condition is satisfied in respect to a particular message, the auto-forwarding method next involves generating and storing in the database, a “forwarding message” that includes at least a portion of the message addressed to the receiver account, and addressed to each member of the “automatic-forwarding client account set” associated with the member of the “automatic-forwarding message category set” (FIG. 3, step 306).

In embodiments, the client may configure additional message category-specific settings stored in the database (12) that control the generation and storage of such forwarding messages. For example, the client may prescribe that messages associated with a particular “message category input” be forwarded within a specified date and/or time range.

As another example, in embodiments, the forwarding message may be stored in association with a message category for the client account that receives the forwarded message (in this context, the “forwardee account”). In an illustrative example, suppose that all client accounts created under the “physician” role have a default permitted message category set (as defined by the administrator) that includes message category “On Call Covering”. Another client account (hereinafter the “forwarder account”) may use a GUI to configure the “automatic-forwarding message category set”, and the associated “automatic-forwarding client account set(s)” as described above. When configuring the “automatic-forwarding client account set”, the forwarder client account holder adds the forwardee account to the “automatic-forwarding client account set”. At that time, the GUI presents a dropdown list of message categories within the forwardee account holder's “permitted message category set”. As an example, if forwarder account is the radiologist #1 account, and radiologist #2 is a member of the “automatic-forwarding client set”, the message category “On Call Covering” may be selected for forwarding messages addressed to the radiologist #2 account.

In one embodiment, upon the forwardee account being added to an “automatic-forwarding client account set”, the system generates a notification (e.g., a message addressed to the forwardee account) to inform the forwardee account holder that he or she has been added to the “automatic-forwarding client account set” with associated details (e.g., the identifier of the forwarder account, the applicable member of the automatic-forwarding message category set, any applicable conditions as the specified date and/or time range for forwarding of messages, and so forth.)

Continuing with the illustrative example, the system (10) generates and stores in the database (12) a first “forwarding message” including message #1, and addressed to the medical office reception account. The system (10) also generates and stores in the database (12) a second “forwarding message” including message #2, and addressed to the radiologist #2 account.

The auto-forwarding method next involves communicating with a client computer device accessing a member of the “automatic-forwarding client account set” to generate on a display device of the client computer device, the forwarding message (FIG. 3, step 306).

Continuing with the illustrative example, the medical office reception account can be used to view the first forwarding message using a mobile app installed on computer device (20d). In this manner, the medical office reception can attend to steps for setting up the requested patient consultation from the GP physician. The radiologist #2 account can be used to view the second forwarding message using a mobile app installed on computer device (20f). In this manner, the radiologist #2 can be apprised of the journal article.

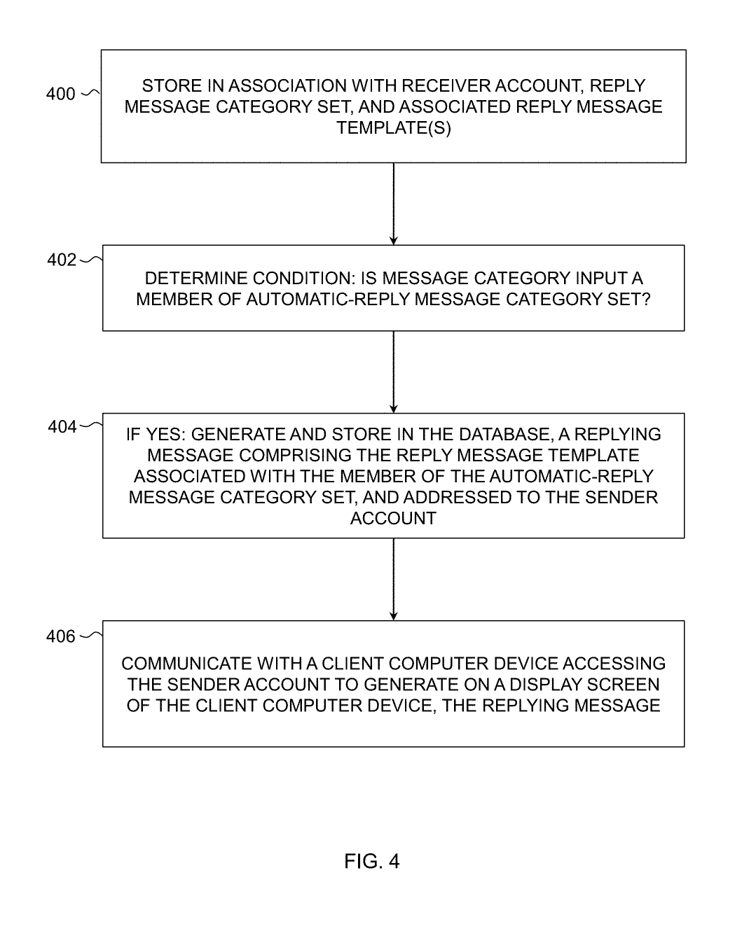

Method for automatic reply to categorized message. FIG. 4 shows optional steps for automatically replying to a message received by the receiver account. These steps allow messages from different categories to be selectively replied (or not replied) to, with different reply messages, with no involvement of the client receiving the message beyond setting automatic reply rules.

The auto-reply method involves storing in the database in association with the receiver account: an “automatic-reply message category set”; and for each member of the automatic-reply message category set, an associated “automatic-reply message template” (FIG. 4, step 400). The “automatic-reply message category set” is a collection of one or more message categories for received messages that are to be replied to the sender account. The “automatic-reply message template” is message content that is used in automatically replying to the sender account.

Continuing with the illustrative example, suppose for instance that the “automatic-reply message category set” for the radiologist #1 account consists of the message category “On-call consults” and “new referrals”. For the message category “On-call consults”, the associated automatic-reply message template consists of the message content “Radiologist #1 is available today for on-call consults only between the hours of 6 pm to 12 am.” For the message category “new referrals”, the associated automatic-reply message template consists of the message content “Radiologist #1 is not accepting new patient referrals.”

The auto-reply method next involves determining if the received message satisfies a condition (FIG. 4, step 402). Minimally, the condition includes that the “message category input” of the received message is a member of the “automatic-reply message category set”. The condition may include other requirements such as the message being received within a specified date and/or time range.

In embodiments, the client may configure the “automatic-reply message category set”, the “automatic-reply message template”, and the condition using a GUI. FIG. 13 shows an illustrative example of such a GUI. The GUI includes a dropdown list labelled “select category” that is populated with message categories that can be selected by the client for adding to the “automatic-reply message category set”. Once one of the categories has been selected, the client can click on the “add button” to display a text input window (lower right of FIG. 13) for creating and saving an associated “automatic-reply message template” in the database (12). The client can view and edit a previously created “automatic-reply message template” by clicking on a “pen and paper” icon to display a text input window (lower left of FIG. 13) for editing the “automatic-reply message template”. The client can also click on the “Delete” button to remove a selected message category from the “automatic-reply message category set”. The GUI includes calendar icons which the client can select to display date/time picker GUIs for specifying the start and end date/times for automatically replying to received messages associated with a particular message category.

Continuing with the illustrative example, suppose that the condition relating to the message category “On-call consults” includes that the message addressed to the receiver account is received between 12 am and 6 pm. Further, suppose that three messages addressed to the radiologist #1 account are stored in the database (12), as follows:
Message #1 is received at 1 pm from the resident account with the “message category input” “On-call consult” and requests a patient consultation. Message #1 satisfies the condition of being within an automatic-reply message category set (as defined above), and being received within a prescribed time range of 12 am to 6 pm. Therefore, message #1 will be automatically replied to;
Message #2 is received from the GP account with the “message category input” “new referral”, for referring a new patient; Message #2 satisfies the condition of being within an automatic-reply message category set (as defined above). Therefore, message #2 will be automatically replied to; and
Message #3 is received from the GP account with the “message category input” “social call”, and pertains to an invitation to a social event. Message #3 does not satisfy the condition of being with the automatic-reply message category set (as defined above). Therefore, message #3 will not be automatically replied to.

When the condition is satisfied in respect to a particular message, the auto-reply method next involves generating and storing in the database, a “replying message” comprising the “reply message template” associated with the member of the “automatic-reply message category set”, and addressed to the sender account (FIG. 4, step 404).

In embodiments, the client may configure message category-specific settings stored in the database (12) that control the generation and storage of such “replying messages”. For example, the client may prescribe that messages associated with a particular “message category input” be replied to within a specified date and/or time range. Also, the client may select the message category under which the replying message is to be sent. (This may be performed using a GUI, in a manner analogous to the manner for selecting the message category under which a forwarding message is to be sent as described above in relation to the automatic-forwarding method.)

Continuing with the illustrative example, the system (10) generates and stores in the database (12) a first “replying message” addressed to the resident account that includes the automatic-reply message template for the “On-call consults” message category. The system (10) also generates and stores in the database (12) a second “replying message” to the GP account that includes the automatic-reply message template for the “new referrals” message category. However, the system (10) does not take any action to reply to the third message.

The auto-reply method next involves communicating with a client computer device accessing the sender account to generate on a display device of the client computer device, the replying message (FIG. 4, step 406).

Continuing with the illustrative example, the resident account can view the first “replying message” using a mobile app installed on computer device (20e). In this manner, the resident is notified that radiologist #1 is unavailable until 6 pm for on-call consultations. The GP account can view the second “replying message” using a web browser installed on computer device (20b). In this manner, the GP is notified that the radiologist #1 is not accepting new patient referrals.

Application to Conventional Email Systems. The foregoing example illustrates the implementation of the present invention in a purpose-built system with dedicated user interfaces, the use of which is restricted to client accounts registered with the system. However, it will be appreciated that conventional email systems may be adapted to implement the present invention, without departing from the scope of invention as defined by the claims.

Additional Interpretation. References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such module, aspect, feature, structure, or characteristic with other embodiments, whether or not explicitly described. In other words, any module, element or feature may be combined with any other element or feature in different embodiments, unless there is an obvious or inherent incompatibility, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a”, “an,” and “the” include the plural reference unless the context clearly dictates otherwise. The term “and/or” means any one of the items, any combination of the items, or all of the items with which this term is associated. The phrase “one or more” is readily understood by one of skill in the art, particularly when read in context of its usage.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio.

1. A method for electronic messaging between client accounts including a sender account and a receiver account, the method implemented using a server computer device operatively connected to a computer-readable database, and a communications interface for communicating via a communication network with computer devices, the method comprising the steps of:
(a) storing in the database in association with the receiver account, a permitted message category set;
(b) communicating with a sender computer device accessing the sender account to generate on a display device of a sender computer device, a message category input graphical user interface (GUI) element configured to receive a message category input limited to a member of the permitted message category set, for association with a message addressed to the receiver account;
(c) receiving from the sender computer device, and storing in the database, the message category input in association with the message addressed to the receiver account; and
(d) communicating with a receiver computer device accessing the receiver account to generate on a display device of the receiver computer device:
(i) for each member of the permitted message category set, a message category GUI element corresponding to a different one of the members of the permitted message category set; and
(ii) a message indicator GUI element indicative of the message addressed to the receiver account stored in the database, in association with the message category GUI element corresponding to the message category input.

2. The method of claim 1, wherein:
(a) the method further comprises the step of: storing in the database in association with the receiver account, for each member of the permitted message category set, an associated permitted sender client account set; and
(b) the message category input GUI element is configured to further limit the message category input to a member of the permitted message category set for which the sender account is a member of the associated permitted sender client account set.

3. The method of claim 1, wherein the permitted message category set is configurable from a computer device accessing the receiver account.

4. The method of claim 2, wherein the permitted sender client account set is configurable from a computer device accessing the receiver account.

5. The method of claim 1, wherein the method further comprises the steps of:
(a) storing in the database in association with the receiver account:
(i) an automatic-forwarding message category set; and
(ii) for each member of the automatic-forwarding message category set, an associated automatic-forwarding client account set;
(b) determining if a condition is satisfied, the condition comprising at least that the message category input corresponds to a member of the automatic-forwarding message category set;
(c) when the condition is satisfied, generating and storing in the database, a forwarding message comprising at least a portion of the message addressed to the receiver account, and addressed to each member of the automatic-forwarding client account set associated with the member of the automatic-forwarding message category set; and
(d) communicating with a client computer device accessing a member of the automatic-forwarding client account set to generate on a display device of the client computer device, the forwarding message.

6. The method of claim 1, wherein the method further comprises the steps of:
(a) storing in the database in association with the receiver account, an automatic-reply message category set; and
(b) determining if a condition is satisfied, the condition comprising at least that the message category input corresponds to a member of the automatic-reply message category set;
(c) when the condition is satisfied, generating and storing in the database, a replying message addressed to the sender account; and
(d) communicating with a client computer device accessing the sender account to generate on a display device of the client computer device, the replying message.

7. The method of claim 6, wherein:
(a) the method further comprises the step of: storing in the database in association with the receiver account, for each member of the automatic-reply message category set, an associated reply message template; and
(b) the replying message comprises the reply message template associated with the member of the automatic-reply message category set.

8. The method of claim 1, wherein:
(a) each message category GUI element comprises a spatially distinct region on the display device of the receiver computer device; and
(b) the message indicator GUI element is associated with the message category GUI element corresponding to the message category input by being disposed within the region corresponding to the message category input.

9. The method of claim 1, wherein:
(a) each message category GUI element comprises an item in a selectable list; and
(b) the message indicator GUI element is associated with the message category GUI element corresponding to the message category input by being displayed when the item corresponding to the message category input is selected from the list.

10. The method of claim 9, wherein the message indicator GUI element comprises one or a combination of an identifier for the sender account, a message subject line, or a date or time corresponding approximately to when the message addressed to the receiver account was stored in the database.

11. The method of claim 1, wherein the communication network comprises the Internet.

12. A system for electronic messaging between client accounts including a sender account and a receiver account, the system comprising:
a server computer device;
a communications interface operatively connected to the server computer device for communicating via a communication network with computer devices;
a computer-readable database operatively connected to the server computer device, and storing in association with the receiver account, a permitted message category set; and
a non-transitory computer readable medium operatively connected to the server computer device, and storing instructions executable by the server computer device to implement a method comprising the steps of:
(a) communicating with a sender computer device accessing the sender account to generate on a display device of a sender computer device, a message category input graphical user interface (GUI) element configured to receive a message category input limited to a member of the permitted message category set, for association with a message addressed to the receiver account;
(b) receiving from the sender computer device, and storing in the database, the message category input in association with the message addressed to the receiver account; and
(c) communicating with a receiver computer device accessing the receiver account to generate on a display device of the receiver computer device:
(i) for each member of the permitted message category set, a message category GUI element corresponding to a different one of the members of the permitted message category set; and
(ii) a message indicator GUI element indicative of the message addressed to the receiver account stored in the database, in association with the message category GUI element corresponding to the message category input.

13. The system of claim 12, wherein:
(a) the database is storing in association with the receiver account, for each member of the permitted message category set, an associated permitted sender client account set; and
(b) the message category input GUI element is configured to further limit the message category input to a member of the permitted message category set for which the sender account is a member of the associated permitted sender client account set.

14. The system of claim 12, wherein the permitted message category set is configurable from a computer device accessing the receiver account.

15. The system of claim 13, wherein the permitted sender client account set is configurable from a computer device accessing the receiver account.

16. The system of claim 12, wherein:
(a) the database is storing in association with the receiver account:
(i) an automatic-forwarding message category set; and
(ii) for each member of the automatic-forwarding message category set, an associated automatic-forwarding client account set; and
(b) the method further comprises the steps of:
(i) determining if a condition is satisfied, the condition comprising at least that the message category input corresponds to a member of the automatic-forwarding message category set;
(ii) when the condition is satisfied, generating and storing in the database, a forwarding message comprising at least a portion of the message addressed to the receiver account, and addressed to each member of the automatic-forwarding client account set associated with the member of the automatic-forwarding message category set; and
(iii) communicating with a client computer device accessing a member of the automatic-forwarding client account set to generate on a display device of the client computer device, the forwarding message.

17. The system of claim 12, wherein:
(a) the database is storing in association with the receiver account, an automatic-reply message category set; and
(b) the method further comprises the steps of:
(i) determining if a condition is satisfied, the condition comprising at least that the message category input corresponds to a member of the automatic-reply message category set;
(ii) when the condition is satisfied, generating and storing in the database, a replying message addressed to the sender account; and
(iii) communicating with a client computer device accessing the sender account to generate on a display device of the client computer device, the replying message.

18. The system of claim 17, wherein:
(a) the database is storing in association with the receiver account, for each member of the automatic-reply message category set, an associated reply message template; and
(b) the replying message comprises the reply message template associated with the member of the automatic-reply message category set.

19. The system of claim 12, wherein:
(a) each message category GUI element comprises a spatially distinct region on the display device of the receiver computer device; and
(b) the message indicator GUI element is associated with the message category GUI element corresponding to the message category input by being disposed within the region corresponding to the message category input.

20. The system of claim 12, wherein:
(a) the message category GUI elements comprise a plurality of items in a selectable list, each of which represents a different one of the plurality of message categories, one of which corresponds to the message category input; and
(b) the message indicator GUI element is associated with the message category GUI element corresponding to the message category input by being displayed when the item representing the message category corresponding to the message category input is selected from the list.

21. The system of claim 20, wherein the message indicator GUI element comprises one or a combination of an identifier for the sender account, a message subject line, or a date or time corresponding approximately to when the message addressed to the receiver account was stored in the database.

22. The system of claim 12, wherein the communication network comprises the Internet, and the system comprises a web server.

23. A program product comprising a non-transitory computer readable medium storing instructions executable by a server computer device operatively connected to a computer-readable database storing in association with the receiver account a permitted message category set, and to a communications interface for communicating via a communications network with computer devices, to implement a method for electronic messaging between client accounts including a sender account and a receiver account, the method comprising the steps of:
(a) communicating with a sender computer device accessing the sender account to generate on a display device of a sender computer device, a message category input graphical user interface (GUI) element configured to receive a message category input limited to a member of the permitted message category set, for association with a message addressed to the receiver account;
(b) receiving from the sender computer device, and storing in the database, the message category input in association with the message addressed to the receiver account; and
(c) communicating with a receiver computer device accessing the receiver account to generate on a display device of the receiver computer device:
(i) for each member of the permitted message category set, a message category GUI element corresponding to a different one of the members of the permitted message category set; and
(ii) a message indicator GUI element indicative of the message addressed to the receiver account stored in the database, in association with the message category GUI element corresponding to the message category input.

24. The program product of claim 23, wherein:
(a) the database is storing in association with the receiver account, for each member of the permitted message category set, an associated permitted sender client account set; and
(b) the message category input GUI element is configured to further limit the message category input to a member of the permitted message category set for which the sender account is a member of the associated permitted sender client account set.

25. The program product of claim 23, wherein the permitted message category set is configurable from a computer device accessing the receiver account.

26. The program product of claim 24, wherein the permitted sender client account set is configurable from a computer device accessing the receiver account.

27. The program product of claim 23, wherein the database is storing in association with the receiver account: an automatic-forwarding message category set; and for each member of the automatic-forwarding message category set, an associated automatic-forwarding client account set; and the method further comprises the steps of:
(a) determining if a condition is satisfied, the condition comprising at least that the message category input corresponds to a member of the automatic-forwarding message category set;
(b) when the condition is satisfied, generating and storing in the database, a forwarding message comprising at least a portion of the message addressed to the receiver account, and addressed to each member of the automatic-forwarding client account set associated with the member of the automatic-forwarding message category set; and
(c) communicating with a client computer device accessing a member of the automatic-forwarding client account set to generate on a display device of the client computer device, the forwarding message.

28. The program product of claim 23, wherein the database is storing in association with the receiver account, an automatic-reply message category set, and the method further comprises the steps of:
(a) determining if a condition is satisfied, the condition comprising at least that the message category input corresponds to a member of the automatic-reply message category set;
(b) when the condition is satisfied, generating and storing in the database, a replying message addressed to the sender account; and
(c) communicating with a client computer device accessing the sender account to generate on a display device of the client computer device, the replying message.

29. The program product of claim 28, wherein:
(a) the database is storing in association with the receiver account, for each member of the automatic-reply message category set, an associated reply message template; and
(b) the replying message comprises the reply message template associated with the member of the automatic-reply message category set.

30. A method for generating an electronic message from a sender account addressed to a receiver account, the method implemented using a sender computer device comprising a display device and in communication via a communication network with a database storing in association with the receiver account, a permitted category message set, the method comprising the steps of:
(a) generating on the display device, a message category input graphical user interface (GUI) element configured to receive a message category input limited to a member of the permitted message category set stored in the database, for association with a message addressed to the receiver account;
(b) using the GUI element to provide a message category input for association with the message addressed to the receiver account; and
(c) initiating transmission to the database for storage, the message category input in association with the message addressed to the receiver account.

31. A method for handling an electronic message from a sender account addressed to a receiver account, the method implemented using a receiver computer device comprising a display device and in communication via a communication network with a database storing the message in association with a message category input limited to a member of a permitted category set stored in the database in association with the receiver account, the method comprising the steps of:
(a) generating on the display device, for each member of the permitted message category set, a message category GUI element corresponding to a different one of the members of the permitted message category set; and
(b) generating on the display device, a message indicator GUI element indicative of the message addressed to the receiver account stored in the database, in association with the message category GUI element corresponding to the message category input.
